(19) United States
(12) Patent Application Publication
(10) Pub. No.: US 2026/0131095 A1
(43) Pub. Date: May 14, 2026

(54) TRACHEAL INTUBATION ASSISTANCE DEVICE AND METHOD FOR REAL-TIME CONFIRMING POSITION CORRECTNESS OF ENDOTRACHEAL TUBE

(71) Applicant: China Medical University, Taichung City (TW)

(72) Inventor: Kai-Sheng Hsieh, Kaohsiung City (TW)

(73) Assignee: China Medical University, Taichung City (TW)

(21) Appl. No.: 19/257,971

(22) Filed: Jul. 2, 2025

(30) Foreign Application Priority Data
Nov. 8, 2024 (TW) 113142948

(51) Int. Cl. A61M 16/04; G06T 7/73

(52) U.S. Cl. CPC A61M 16/0402; A61M 2205/3561; A61M 2205/502; A61M 2205/58; A61M 2210/1032; G06T 7/74; G06T 2207/10048; G06T 2207/30004

(57) ABSTRACT

A tracheal intubation assistance device and a method for real-time confirming a position correctness of an endotracheal tube are provided. The method for real-time confirming the position correctness of the endotracheal tube includes providing the tracheal intubation assistance device, performing an image capture step, performing a region selection step, and performing an image interpretation step. The tracheal intubation assistance device includes an infrared temperature sensor and a processor. The infrared temperature sensor is configured to capture a thermal image of a workspace including the endotracheal tube and a subject, and the processor is configured to compare a first temperature range of a first sampling region with a second temperature range of a second sampling region to confirm the position correctness of the endotracheal tube.

### RELATED APPLICATIONS

This application claims priority to Taiwan Application Serial Number 113142948, filed Nov. 8, 2024, which is herein incorporated by reference.

### BACKGROUND

### Technical Field

The present disclosure relates to a tracheal intubation assistance device and a method for confirming a position correctness of an endotracheal tube. More particularly, the present disclosure relates to a tracheal intubation assistance device and a method for real-time confirming a position correctness of an endotracheal tube using a thermal image.

### Description of Related Art

Tracheal intubation is a procedure performed when respiratory failure occurs, in which an endotracheal tube is inserted through the upper respiratory tract into the trachea to establish a clear and unobstructed airway. This facilitates connection to a ventilator for breathing support and oxygen delivery, as well as assists in clearing mucus from the respiratory tract via the endotracheal tube.

During tracheal intubation, a medical professional first opens the airway of the patient and uses a laryngoscope to lift the tongue and visualize the tracheal opening as much as possible. Since the esophagus is positioned directly behind the trachea, misplacement of the endotracheal tube into the esophagus is a common occurrence. Because the human body is not a closed system, oxygen mistakenly delivered into the esophagus may only cause gastrointestinal distension and can be expelled through the mouth and nose. However, incorrect positioning of the endotracheal tube can delay treatment and seriously affect the patient's life safety.

Accurately and successfully inserting the endotracheal tube into the trachea typically relies on the expertise of medical professionals, who confirm endotracheal tube position by observing chest movement or auscultating the chest and stomach. In some situations, medical professionals may have difficulty accurately confirming the position of the endotracheal tube, which can lead to incorrect confirmation and inadvertent insertion into the esophagus. Additionally, the need for repeated observations may be time-consuming, potentially delaying emergency treatment. Measuring the patient's blood oxygen saturation levels can accurately confirm the positon of the endotracheal tube, but this method is time-consuming. Observing condensation inside the endotracheal tube during ventilation is another potential method, but it is not applicable in many emergency situations. Conventional devices for measuring carbon dioxide concentration to confirm the correctness of the position of the endotracheal tube usually exist as a stand-alone device, or as a module to connect the endotracheal tube to a monitor to monitor the carbon dioxide concentration. This method requires continuous measurement of the carbon dioxide concentration of several breaths to confirm the result, which is also time-consuming and may delay emergency treatment.

### SUMMARY

According to one embodiment of the present disclosure, a tracheal intubation assistance device, which is used in conjunction with an endotracheal tube to provide a real-time assistance in confirming a position correctness of the endotracheal tube, includes an infrared temperature sensor and a processor. The infrared temperature sensor is configured to capture a thermal image of a workspace, wherein the workspace includes the endotracheal tube and a subject. The processor is electrically connected to the infrared temperature sensor and includes a display module, a selection module, and a comparison module. The display module is configured to display the thermal image. The selection module is configured to select a first sampling region and a second sampling region from the thermal image, wherein the first sampling region is located at the endotracheal tube and the second sampling region is located at a facial area of the subject. The comparison module is configured to compare a first temperature range of the first sampling region with a second temperature range of the second sampling region to obtain a confirmation result of the position correctness. When the first temperature range is the same as the second temperature range, the confirmation result is that a position of the endotracheal tube is correct. When the first temperature range is lower than the second temperature range, the confirmation result is that the position of the endotracheal tube is incorrect.

According to another embodiment of the present disclosure, a method for real-time confirming a position correctness of an endotracheal tube includes providing the aforementioned tracheal intubation assistance device, performing an image capture step, performing a region selection step, and performing an image interpretation step. In the image capture step, the infrared temperature sensor captures a thermal image of a workspace, wherein the workspace includes an endotracheal tube and a subject. In the region selection step, the display module displays the thermal image, and the selection module selects a first sampling region and a second sampling region from the thermal image, wherein the first sampling region is located at the endotracheal tube, and the second sampling region is located at a facial area of the subject. In the image interpretation step, the comparison module compares the first temperature range of the first sampling region with the second temperature range of the second sampling region to obtain a confirmation result of the position correctness of the endotracheal tube. When the first temperature range is the same as the second temperature range, the confirmation result is that a position of the endotracheal tube is correct. When the first temperature range is lower than the second temperature range, the confirmation result is that the position of the endotracheal tube is incorrect.

### BRIEF DESCRIPTION OF THE DRAWINGS

The present disclosure can be more fully understood by reading the following detailed description of the embodiment, with reference made to the accompanying drawings as follows:

FIG. 1 is a schematic view of a tracheal intubation assistance device according to one embodiment of the present disclosure.

FIG. 2 is structural schematic view of an infrared temperature sensor in the tracheal intubation assistance device of FIG. 1.

FIG. 3 is a step flow chart of a method for real-time confirming a position correctness of an endotracheal tube according to another embodiment of the present disclosure.

FIG. 4 is a schematic view showing a correct position of an endotracheal tube.

FIG. 5 is a thermal image of a workspace captured by the infrared temperature sensor.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. Moreover, for the sake of simplicity, some conventional structures and components will be depicted schematically in the drawings and repetitive components can be represented by the same reference numbers.

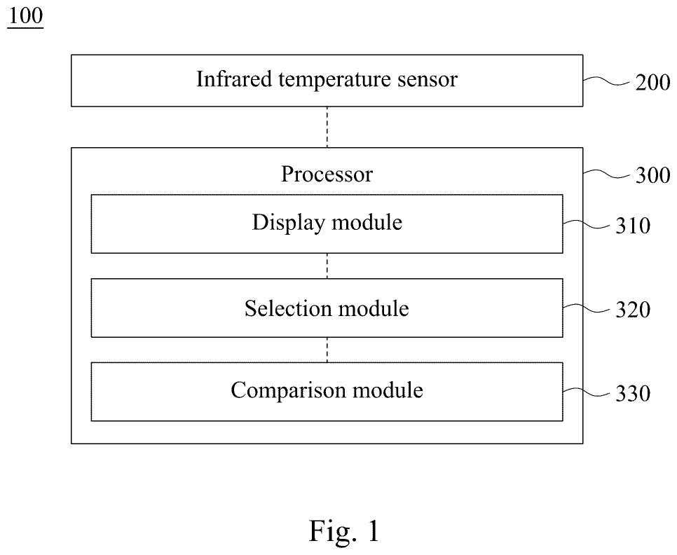

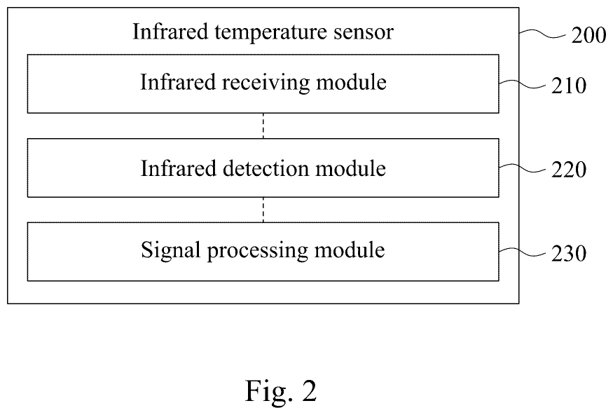

Reference is made to FIG. 1 and FIG. 2, FIG. 1 is a schematic view of a tracheal intubation assistance device 100 according to one embodiment of the present disclosure, and FIG. 2 is structural schematic view of an infrared temperature sensor 200 in the tracheal intubation assistance device 100 of FIG. 1. The tracheal intubation assistance device 100 is used in conjunction with an endotracheal tube (not shown) to provide a real-time assistance in confirming a position correctness of the endotracheal tube. The tracheal intubation assistance device 100 includes an infrared temperature sensor 200 and a processor 300.

The infrared temperature sensor 200 is configured to capture a thermal image of a workspace, wherein the workspace includes the endotracheal tube and a subject. The infrared temperature sensor 200 can be a thermal imager or an infrared camera. Furthermore, the infrared temperature sensor 200 can include an infrared receiving module 210, an infrared detection module 220, and a signal processing module 230. The infrared receiving module 210 is configured to receive an infrared radiation spectrum in the workspace and transmit it to the infrared detection module 220. The infrared detection module 220 is configured to convert the infrared radiation spectrum into an electrical signal. The signal processing module 230 is configured to receive the electrical signal and perform a processing calculation to obtain the thermal image of the workspace.

Specifically, the infrared receiving module 210 captures the infrared radiation spectrum emitted by objects in the workspace, that is, the infrared light emitted by the objects, including the infrared light emitted by the endotracheal tube and the ventilation flow caused by the breathing of the subject in the workspace. Furthermore, the infrared receiving module 210 can also collect the infrared radiation spectrum, and the infrared receiving module 210 can be an infrared lens. When the infrared radiation spectrum emitted by the objects in the workspace is received and converged by the infrared lens, it is directly transmitted to the infrared detection module 220.

The infrared detection module 220 can include an infrared light absorber (not shown), a thermoelectric element (not shown), and an electrical signal detection element (not shown). The infrared light absorber is disposed on the thermoelectric element and is directly in contact with the thermoelectric element, and the electrical signal detection element is electrically connected to the thermoelectric element through a wire. The electrical signal detection element and the thermoelectric element are connected in series to form a loop for detecting the electrical signal change of the thermoelectric element. Accordingly, the infrared detection module 220 can convert the infrared radiation spectrum received from the infrared receiving module 210 into the electrical signal, and then transmits the electrical signal to the signal processing module 230.

The signal processing module 230 receives the electrical signal and performs the processing calculation to obtain the thermal field distribution of the object. Specifically, the signal processing module 230 calculates the temperature data corresponding to surface position of the object based on variations in the electrical signal from the infrared detection module 220 to obtain the thermal image of the workspace. Therefore, the signal processing module 230 can calculate the thermal field distribution data of the object bases on the electrical signal to obtain the thermal image indicating different temperature ranges, wherein different temperatures are represented in different colors. As a result, the thermal image corresponds to the temperature distribution of the object, reflecting the temperature conditions at various positions of the object. That is, in the obtained thermal image of the workspace, the thermal field distribution data of the endotracheal tube and the subject are marked, providing a representation of the temperature conditions of the endotracheal tube and the subject at different positions within the workspace.

The processor 300 is electrically connected to the infrared temperature sensor 200 and includes a display module 310, a selection module 320, and a comparison module 330. The display module 310 is configured to display the thermal image. The selection module 320 is configured to select a first sampling region and a second sampling region from the thermal image, wherein the first sampling region is located at the endotracheal tube and the second sampling region is located at a facial area of the subject. The comparison module 330 is configured to compare a first temperature range of the first sampling region with a second temperature range of the second sampling region to obtain a confirmation result of the position correctness. When the first temperature range is the same as the second temperature range, the confirmation result is that a position of the endotracheal tube is correct. When the first temperature range is lower than the second temperature range, the confirmation result is that the position of the endotracheal tube is incorrect.

Furthermore, the processor 300 can include a reminder module (not shown), which is configured to send a reminder signal. The reminder signal can be a light signal, a sound signal or a vibration signal. Additionally, the processor 300 can include a wireless transmission module (not shown), which is configured to transmit the confirmation result to a terminal device.

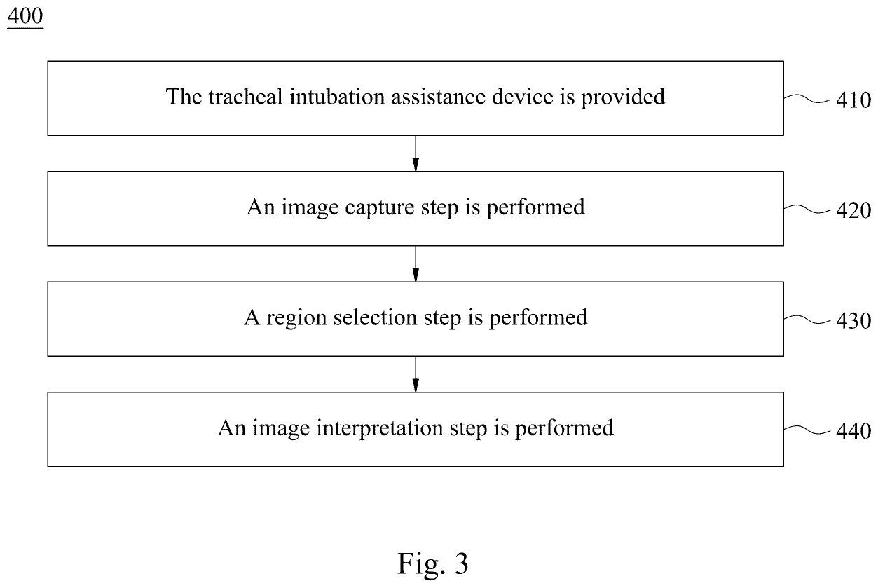

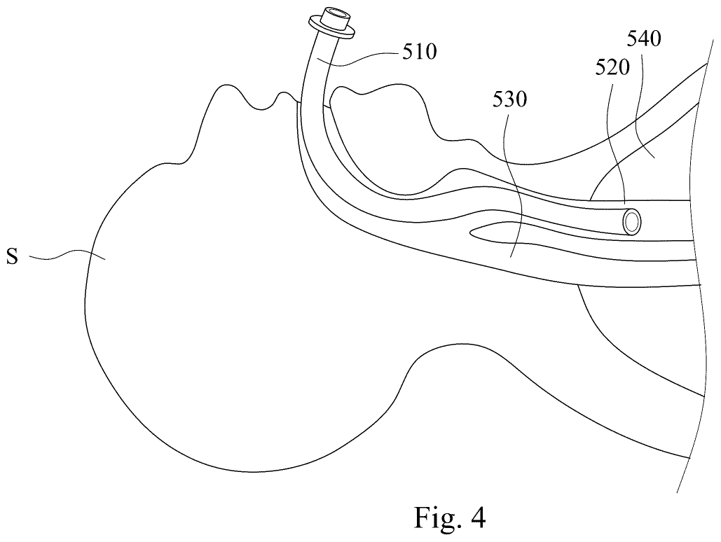

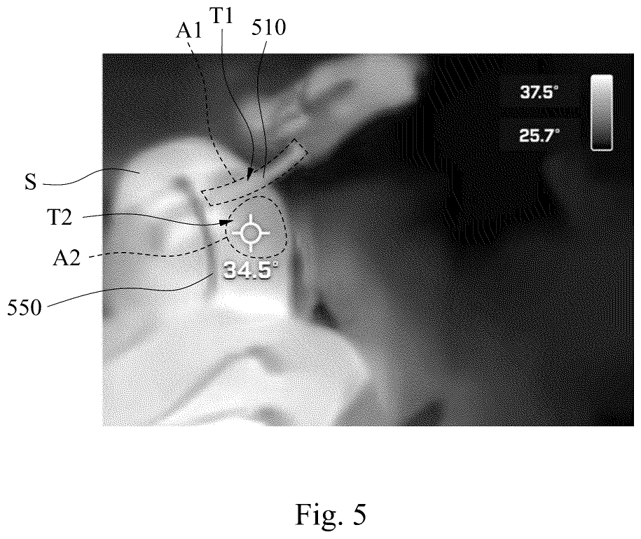

Reference is made to FIG. 3 to FIG. 5. FIG. 3 is a step flow chart of a method for real-time confirming a position correctness of an endotracheal tube 400 according to another embodiment of the present disclosure. FIG. 4 is a schematic view showing a correct position of an endotracheal tube 510. FIG. 5 is a thermal image of a workspace captured by the infrared temperature sensor 200. The method for real-time confirming the position correctness of the endotracheal tube 400 includes Step 410, Step 420, Step 430, and Step 440.

In Step 410, the tracheal intubation assistance device 100 is provided. The tracheal intubation assistance device 100 includes the infrared temperature sensor 200 and the processor 300, and the processor 300 is electrically connected to the infrared temperature sensor 200.

In Step 420, an image capture step is performed, in which the infrared temperature sensor 200 captures a thermal image of a workspace, and the workspace includes an endotracheal tube 510 and a subject S.

In Step 430, a region selection step is performed, in which the display module 310 displays the thermal image, and the selection module 320 selects a first sampling region A1 and a second sampling region A2 from the thermal image. The first sampling region A1 is located at the endotracheal tube 510, and the second sampling region A2 is located at a facial area of the subject S.

In Step 440, an image interpretation step is performed, in which the comparison module 330 compares a first temperature range T1 of the first sampling region A1 with a second temperature range T2 of the second sampling region A2 to obtain a confirmation result of the position correctness of the endotracheal tube 510. When the first temperature range T1 is the same as the second temperature range T2, the confirmation result is that a position of the endotracheal tube 510 is correct. When the first temperature range T1 is lower than the second temperature range T2, the confirmation result is that the position of the endotracheal tube 510 is incorrect. The second temperature range T2 can be 32° C. to 39° C.

Furthermore, the processor 300 can include a reminder module, which is configured to send a reminder signal when the confirmation result is that the position of the endotracheal tube 510 is incorrect. The reminder signal can be a light signal, a sound signal or a vibration signal. The processor 300 can also include a wireless transmission module, which is configured to transmit the confirmation result to a terminal device for remote monitoring after the confirmation result is obtained in the image interpretation step.

As shown in FIG. 4, when intubating the trachea 520, the medical professional inserts the endotracheal tube 510 through the oral cavity or nasal cavity, passing through the throat, glottis and vocal cords before entering the trachea 520. The endotracheal tube 510 is then fixed at a position 1 cm to 2.5 cm beyond the vocal cords. When observed at the glottis, the trachea 520 and esophagus 530 appear as two nested ovals, with the esophagus 530 positioned directly behind the trachea 520. If the position of the endotracheal tube 510 is correct, the endotracheal tube 510 is intubated into the trachea 520 and can be connected to the lung 540. The air passing through the endotracheal tube 510 during the respiration of the subject S will have a temperature close to body temperature. Consequently, when the position of the endotracheal tube 510 is correct, in the thermal image of the workspace captured by the infrared temperature sensor 200, the first temperature range T1 of the first sampling region A1 located at the endotracheal tube 510 is the same as the second temperature range T2 of the second sampling region A2 located at the facial area of the subject S. If the endotracheal tube 510 is incorrectly inserted into the esophagus 530, a small amount of gas flowing in the endotracheal tube 510 comes from the gastrointestinal tract, with a temperature range similar to ambient temperature such as room temperature. Consequently, when the position of the endotracheal tube 510 is incorrect, in the thermal image of the workspace captured by the infrared temperature sensor 200, the first temperature range T1 of the first sampling region A1 located at the endotracheal tube 510 is lower than the second temperature range T2 of the second sampling region A2 located at the facial area of the subject S.

Reference is made to FIG. 5. In this embodiment, the correct position of the endotracheal tube 510 in the subject S is initially confirmed through auscultation of the stomach, bilateral lung bases, and bilateral lung apices, followed by secondary confirmation using a tidal end-tidal carbon dioxide (ET-CO₂) monitor. In FIG. 5, the thermal image of the workspace shows different temperature ranges in different lightness. As shown in FIG. 5, the second temperature range T2 of the second sampling region A2 located at the facial area of the subject S is approximately 34.5° C. When the position of the endotracheal tube 510 is correct, the first temperature range T1 of the first sampling region A1 located at the endotracheal tube 510 and the second temperature range T2 of the second sampling region A2 located at the facial area of the subject S are the same. In the thermal image of the workspace, the nasogastric tube 550 is not directly connected to the lung 540. As the result, no gas at body temperature flows in or out, causing the temperature range displayed for the nasogastric tube 550 to be close to the ambient temperature.

In summary, the tracheal intubation assistance device of the present disclosure, including only an infrared temperature sensor and processor, is compact, portable, and requires minimal power, offering high mobility for various emergency settings to assist in real-time confirming a position correctness of the endotracheal tube. The method for real-time confirming the position correctness of the endotracheal tube of the present disclosure can quickly and accurately obtain a confirmation result of the position correctness of the endotracheal tube by capturing a thermal image of the workspace including the endotracheal tube and the subject, selecting a first sampling region located at the endotracheal tube and a second sampling region located at a facial area of the subject from the thermal image, and then comparing a first temperature range of the first sampling region with a second temperature range of the second sampling region. Therefore, the method for real-time confirming the position correctness of the endotracheal tube of the present disclosure is non-invasive and enables rapid and accurate real-time confirmation of the position of the endotracheal tube. By eliminating delays caused by conventional multi-step procedures involving primary and secondary assessments, the method for real-time confirming the position correctness of the endotracheal tube of the present disclosure maximizes the critical golden period for emergency care, thereby improving the success rate of resuscitation efforts.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A tracheal intubation assistance device, which is used in conjunction with an endotracheal tube to provide a real-time assistance in confirming a position correctness of the endotracheal tube, wherein tracheal intubation assistance device comprises:
an infrared temperature sensor configured to capture a thermal image of a workspace, wherein the workspace comprises the endotracheal tube and a subject; and
a processor electrically connected to the infrared temperature sensor, wherein the processor comprises:
a display module configured to display the thermal image;
a selection module configured to select a first sampling region and a second sampling region from the thermal image, wherein the first sampling region is located at the endotracheal tube and the second sampling region is located at a facial area of the subject; and
a comparison module configured to compare a first temperature range of the first sampling region with a second temperature range of the second sampling region to obtain a confirmation result of the position correctness;
wherein when the first temperature range is the same as the second temperature range, the confirmation result is that a position of the endotracheal tube is correct;
wherein when the first temperature range is lower than the second temperature range, the confirmation result is that the position of the endotracheal tube is incorrect.

2. The tracheal intubation assistance device of claim 1, wherein the infrared temperature sensor comprises:
an infrared receiving module configured to receive an infrared radiation spectrum in the workspace;
an infrared detection module configured to convert the infrared radiation spectrum into an electrical signal; and
a signal processing module configured to receive the electrical signal and perform a processing calculation to obtain the thermal image of the workspace.

3. The tracheal intubation assistance device of claim 1, wherein the processor further comprises a reminder module configured to send a reminder signal.

4. The tracheal intubation assistance device of claim 3, wherein the reminder signal is a light signal, a sound signal or a vibration signal.

5. The tracheal intubation assistance device of claim 1, wherein the processor further comprises a wireless transmission module configured to transmit the confirmation result to a terminal device.

6. A method for real-time confirming a position correctness of an endotracheal tube, comprising:
providing the tracheal intubation assistance device of claim 1;
performing an image capture step, wherein the infrared temperature sensor captures a thermal image of a workspace, and the workspace comprises an endotracheal tube and a subject;
performing a region selection step, wherein the display module displays the thermal image, the selection module selects a first sampling region and a second sampling region from the thermal image, the first sampling region is located at the endotracheal tube, and the second sampling region is located at a facial area of the subject; and
performing an image interpretation step, wherein the comparison module compares a first temperature range of the first sampling region with a second temperature range of the second sampling region to obtain a confirmation result of the position correctness of the endotracheal tube;
wherein when the first temperature range is the same as the second temperature range, the confirmation result is that a position of the endotracheal tube is correct;
wherein when the first temperature range is lower than the second temperature range, the confirmation result is that the position of the endotracheal tube is incorrect.

7. The method for real-time confirming the position correctness of the endotracheal tube of claim 6, wherein the second temperature range is 32° C. to 39° C.

8. The method for real-time confirming the position correctness of the endotracheal tube of claim 6, wherein the processor further comprises a reminder module configured to send a reminder signal when the confirmation result is that the position of the endotracheal tube is incorrect.

9. The method for real-time confirming the position correctness of the endotracheal tube of claim 8, wherein the reminder signal is a light signal, a sound signal or a vibration signal.

10. The method for real-time confirming the position correctness of the endotracheal tube of claim 9, wherein the processor further comprises a wireless transmission module configured to transmit the confirmation result to a terminal device for remote monitoring after the confirmation result is obtained in the image interpretation step.
